(19) United States
(12) Patent Application Publication
(10) Pub. No.: US 2026/0354878 A1
(43) Pub. Date: Nov. 12, 2026

(54) INSTRUMENT FOR SETTING A LIMITING ELEMENT, AND CORRESPONDING SYSTEM AND KIT

(71) Applicant: MATHYS AG BETTLACH, Bettlach (CH)

(72) Inventors: Luca D'ANTONI, Bettlach (CH); Carina SCHINEIS, Bettlach (CH); Ulrike MÜLLER, Bettlach (CH); Patrick MEYER, Bettlach (CH)

(21) Appl. No.: 19/487,020

(22) PCT Filed: May 30, 2024
(86) PCT No.: PCT/IB2024/055316

§ 371 Date: Nov. 24, 2025

(30) Foreign Application Priority Data
May 31, 2023 (DE) 10 2023 114 190.2

(51) Int. Cl. A61B 17/88; A61F 2/30

(52) U.S. Cl. CPC A61B 17/8802; A61F 2/30723

(57) ABSTRACT

The disclosure relates to an instrument for setting a limiting element for limiting the spread of bone cement. The instrument includes a grip region for gripping the instrument, a hollow shaft having a first end and a second end, a holding region configured to have a detachable connection to the limiting element. The hollow shaft has at least one bend. The holding region is coupled to the second end of the shaft. The instrument further includes a pulling element disposed within an interior of the hollow shaft, and a detaching element disposed in the grip region. The pulling element extends from the grip region to the holding region. The detaching element is configured to detach the detachable connection by means of the pulling element.

### CROSS-REFERENCE TO RELATED APPLICATIONS

This application claims priority to German Patent Application No. 10 2023 114 190.2, filed on May 31, 2023, which is hereby incorporated by reference herein in its entirety.

### FIELD OF DISCLOSURE

The disclosure relates to an instrument for setting a limiting element for limiting the spread of bone cement, a corresponding system comprising such an instrument and such a limiting element, and a kit comprising such an instrument and correspondingly different shafts for the instrument.

### BACKGROUND

Due to an increasing life expectancy of people, the probability or necessity of replacing at least part of a joint with an appropriate implant at least once in the course of a lifetime is also growing. However, in particular when cement or bone cement is used within the scope of the corresponding implantation, it can have a negative effect on the health of the respective patient if the bone cement spreads too far or penetrates too deeply into the corresponding bone. Thus, there is a growing need for options or, for example, limiting elements which allow an appropriate limitation of the spread or penetration depth of the bone cement.

However, there is currently a lack of instruments with which it is possible in a cost-efficient manner to set such limiting elements in a particularly convenient and safe manner.

### OBJECT AND SOLUTION

It is therefore an object of the disclosure to provide an instrument for setting a limiting element for limiting the spread of bone cement, a corresponding system, and a corresponding kit, wherein a particularly convenient and safe setting of the limiting element can be achieved in a cost-effective manner.

The object is achieved with respect to the instrument for setting a limiting element for limiting the spread of bone cement by the features of claim 1. The object is achieved with respect to the corresponding system comprising such an instrument and such a limiting element by the features of claim 10. The object is achieved with respect to the corresponding kit comprising such an instrument and correspondingly different shafts for the instrument by the features of claim 13. The dependent claims contain advantageous further developments.

### DETAILED DESCRIPTION OF THE DISCLOSURE

In accordance with a first aspect of the disclosure, there is created an instrument for setting a limiting element for limiting the spread of bone cement. The instrument has a grip region for gripping the instrument, a shaft having a first end and a second end, and a holding region for holding the limiting element. The first end of the shaft can be arranged and/or is arranged on the grip region. The holding region can be arranged and/or is arranged at the second end of the shaft. In addition, the shaft has at least one bend and/or curvature.

Advantageously, a particularly convenient and safe setting of the limiting element can thus be achieved in a cost-effective manner.

In accordance with a first implementation of the first aspect of the disclosure, the shaft has at least two shaft portions, preferably at least one straight shaft portion and at least one bent and/or curved shaft portion, particularly preferably three shaft portions, very particularly preferably two straight shaft portions and one bent and/or curved shaft portion, each of which, in particular, can be arranged and/or are arranged on one another.

Advantageously, for example, convenience and safety can thus be further increased since the shaft can be flexibly adapted to the corresponding conditions.

In accordance with a second implementation of the first aspect of the disclosure, the corresponding arrangement of the at least two shaft portions on one another has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

Advantageously, the shaft can thus be adapted in a particularly simple and cost-effective manner, for example.

In accordance with a further implementation of the first aspect of the disclosure, the corresponding arrangement of the first end of the shaft on the grip region has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

Advantageously, this enables, for example, easy cleaning of the instrument, which can also apply analogously to the following implementation.

In accordance with a further implementation of the first aspect of the disclosure, the corresponding arrangement of the holding region at the second end of the shaft has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

In accordance with a further implementation of the first aspect of the disclosure, the shaft has at least one bending radius, preferably exactly one bending radius.

Advantageously, the instrument can thus be manufactured in a simple and cost-effective manner, for example.

In accordance with a further implementation of the first aspect of the disclosure, the at least one bend and/or curvature includes an angle between 100 degrees and 170 degrees, preferably between 110 degrees and 160 degrees, particularly preferably between 120 degrees and 150 degrees, very particularly preferably between 130 degrees and 140 degrees.

Advantageously, for example, even regions that are generally difficult to access can be reached in a convenient and safe manner by means of the limiting element.

In accordance with a further implementation of the first aspect of the disclosure, the holding region has a detachable connection, preferably a plug connection and/or a screw connection and/or a bayonet-type connection, in particular a bayonet connection, which is designed to hold the limiting element.

Advantageously, for example, an unintentional loss of the limiting element can thus be avoided in a reliable manner.

In accordance with a further implementation of the first aspect of the disclosure, the holding region is designed to be rotationally symmetrical. Additionally or alternatively, the holding region has at least one thickening and/or at least one O-Ring, in particular for frictionally holding the limiting element.

Advantageously, for example, not only can an involuntary loss of the limiting element be reliably avoided, but also a deliberate detaching of the limiting element can be simplified.

In accordance with a second aspect of the disclosure, a system is created. The system has an instrument according to the first aspect of the disclosure or one of its implementations, and a limiting element for limiting the spread of bone cement.

Advantageously, a particularly convenient and safe setting of the limiting element can thus be achieved in a cost-effective manner.

In accordance with a first implementation of the second aspect of the disclosure, the limiting element has an interface region which is designed in particular to interact with the holding region of the instrument.

Advantageously, the interface region of the limiting element and the holding region of the instrument can interact, for example, in the sense of a frictional engagement.

In accordance with a second implementation of the second aspect of the disclosure, the limiting element, in particular in the region of the interface region, has at least one predetermined breaking point, preferably exactly one predetermined breaking point.

Advantageously, the at least one or exactly one predetermined breaking point, preferably in the event that the setting of the limiting element is completed, can be designed such that it breaks before the interface region can be detached or is detached from the holding region of the instrument, in particular when the instrument is removed.

In accordance with a third aspect of the disclosure, a kit is created. The kit has an instrument according to the first aspect of the disclosure or one of its implementations and at least two shafts, each having a different bend and/or curvature.

Advantageously, a particularly convenient and safe setting of the limiting element can thus be achieved in a cost-effective manner.

### DESCRIPTION OF PREFERRED IMPLEMENTATIONS

In the following, a detailed exemplary description of some implementations of the disclosure is given with reference to the figures of the drawing. In the drawings:

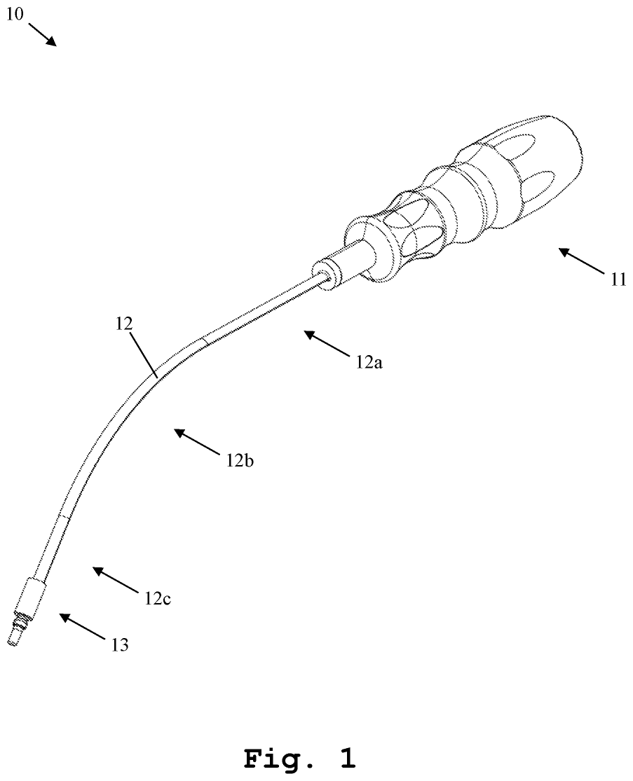

FIG. 1 shows a three-dimensional view of an exemplary implementation of the instrument according to the disclosure;

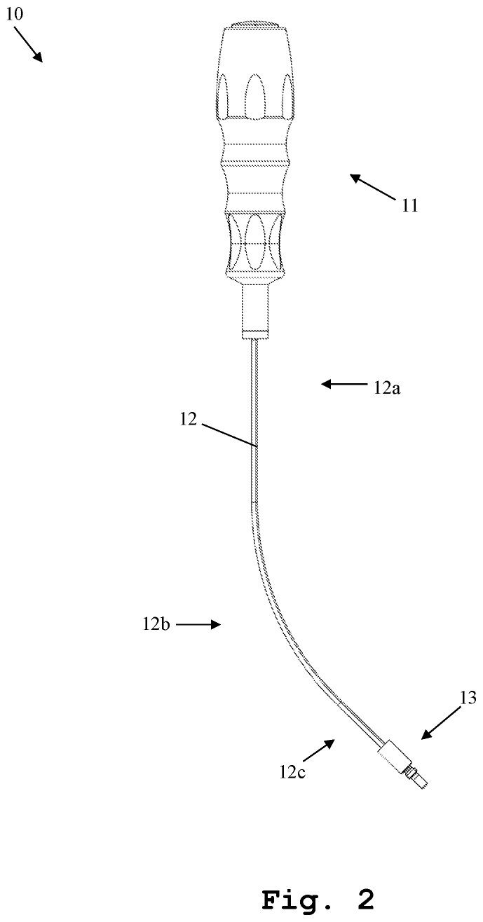

FIG. 2 shows a side view of the implementation in accordance with FIG. 1;

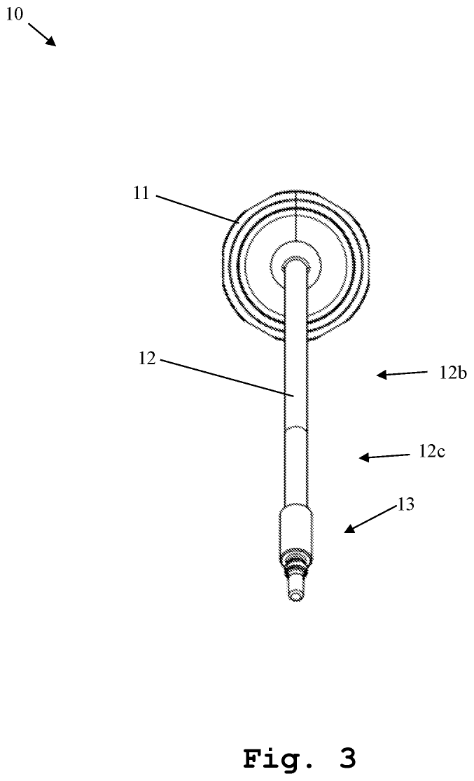

FIG. 3 shows a front view of the implementation in accordance with FIG. 1;

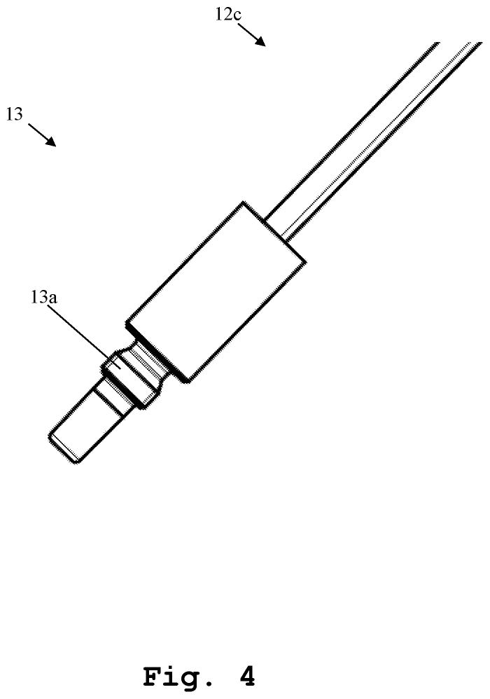

FIG. 4 shows an enlarged view of the holding region of the implementation in accordance with FIG. 1;

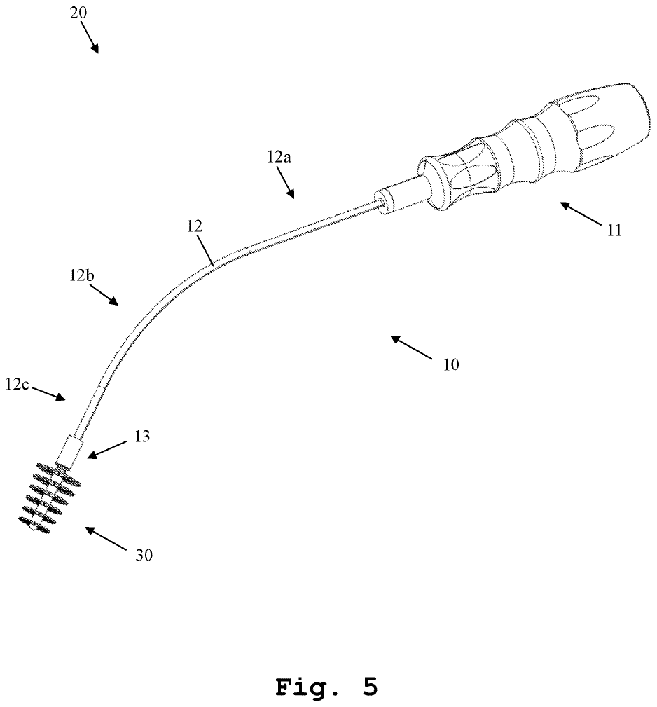

FIG. 5 shows a three-dimensional view of an exemplary implementation of the system according to the disclosure with the instrument in accordance with FIG. 1;

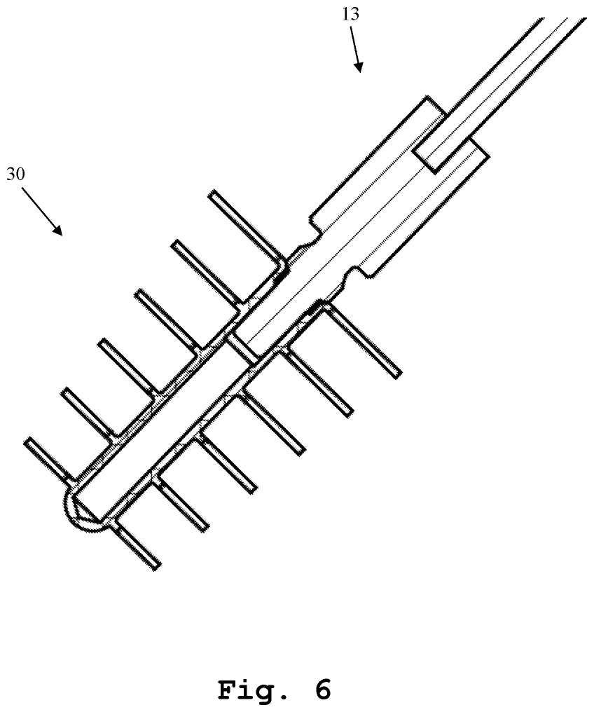

FIG. 6 shows an enlarged sectional view of the system in accordance with FIG. 5 with respect to the holding region of the instrument and of the limiting element;

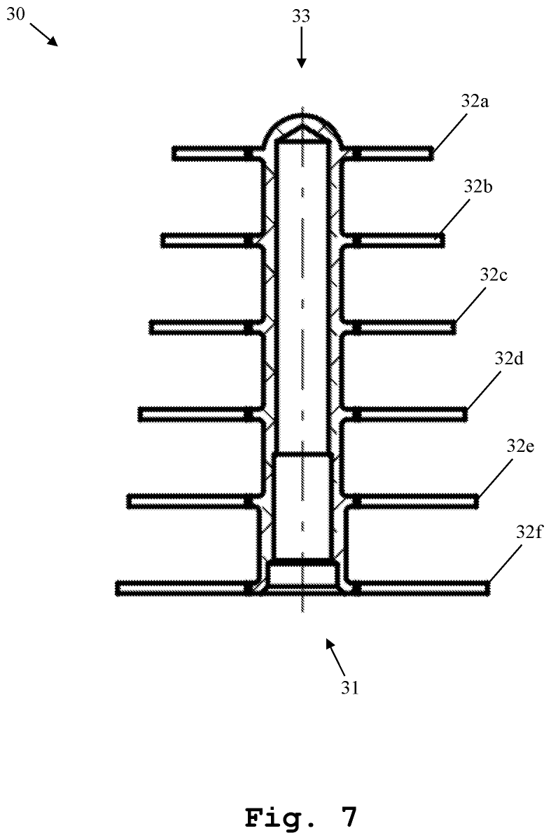

FIG. 7 shows a sectional view of the limiting element of the system in accordance with FIG. 5; and

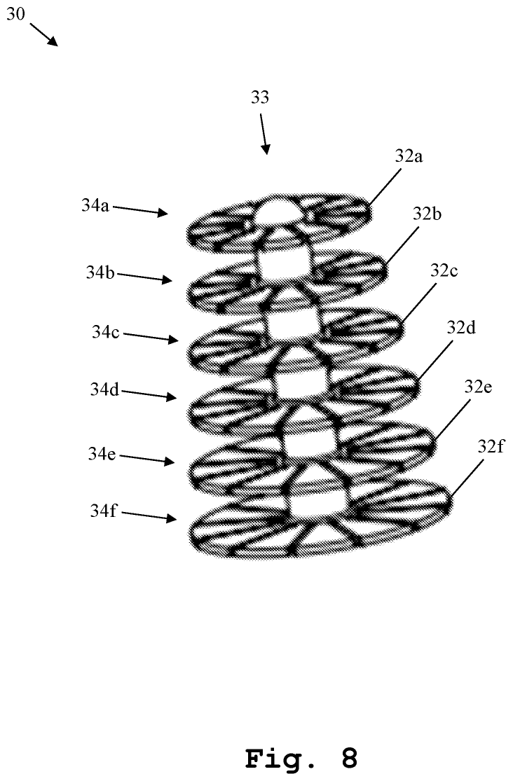

FIG. 8 shows a three-dimensional view of the limiting element of the system in accordance with FIG. 5.

FIG. 1, FIG. 2 and FIG. 3 illustrate a three-dimensional view, a side view, and a front view of an exemplary implementation 10 of an instrument according to the disclosure for setting a limiting element for limiting the spread of bone cement.

The instrument 10 has a grip region 11 which is designed to be gripped by a person. This grip region 11 is preferably designed to be at least partially rotationally symmetrical.

Furthermore, the instrument 10 has a shaft having a first end and a second end and a holding region 13. The holding region 13 is designed to hold the limiting element.

In addition, the first end of the shaft 12 can be arranged and/or is arranged on the grip region 11, while the holding region 13 can be arranged and/or is arranged at the second end of the shaft 12. In addition, the shaft 12 has at least one bend and/or curvature.

It can be advantageous if the shaft 12 has at least two shaft portions, preferably at least one straight shaft portion and at least one bent and/or curved shaft portion, particularly preferably three shaft portions, very particularly preferably two straight shaft portions and one bent and/or curved shaft portion, each of which, in particular, can be arranged and/or are arranged on one another.

According to the example, the shaft 12 has a first shaft portion 12a, which is designed to be straight, a second shaft portion 12b, which is designed to be bend and/or curved, and a third shaft portion 12c, which is designed to be straight.

Here, a first end of the first shaft portion 12a can be arranged and/or is arranged on the grip region. In other words, the first shaft portion 12a has the first end of the shaft 12, which in particular corresponds to the first end of the first shaft portion 12a.

Furthermore, a first end of the second shaft portion 12b can be arranged and/or is arranged at a second end of the first shaft portion 12a, while a first end of the third shaft portion 12c can be arranged and/or is arranged at a second end of the second shaft portion 12b.

In addition, the holding region 13 can be arranged and/or is arranged at a second end of the third shaft portion 12c. In other words, the third shaft portion 12c has the second end of the shaft 12, which in particular corresponds to the second end of the third shaft portion 12c.

With regard to the arrangement of the second shaft portion 12b on the first shaft portion 12a, it should be mentioned that it may be advantageous if this arrangement has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

Regarding the arrangement of the third shaft portion 12c on the second shaft portion 12b, it should be noted that it may be advantageous if this arrangement has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

Further, it may be advantageous if the arrangement of the first end of the shaft 12 on the grip region 11 or the arrangement of the first end of the first shaft portion 12a on the grip region 11 has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

According to the example, the arrangement of the first end of the shaft 12 on the grip region 11 or the arrangement of the first end of the first shaft portion 12a is integrally formed on the grip region 11.

Furthermore, it may be advantageous if the arrangement of the holding region 13 at the second end of the shaft 12 or the arrangement of the holding region 13 at the second end of the third shaft portion 12c has a detachable connection, preferably a plug connection and/or a screw connection and/or a bayonet-type connection, in particular a bayonet connection, or a fixed connection, preferably an adhesive connection and/or a welded connection and/or a rivet connection and/or an integral connection.

According to the example, the arrangement of the holding region 13 at the second end of the shaft 12 or the arrangement of the holding region 13 at the second end of the third shaft portion 12c is integrally formed.

It can also be seen that both the first shaft portion 12a and the third shaft portion 12c are each designed to be rotationally symmetrical in the exemplary implementation. In principle, it may be advantageous if straight shaft portions are each designed to be rotationally symmetrical.

It should be noted that the shaft 12 may have at least one bending radius, preferably exactly one bending radius. According to the example, the bent and/or curved shaft portion 12b has at least one bending radius, preferably exactly one bending radius.

It can be advantageous if the at least one bend and/or curvature or the corresponding bend and/or curvature of the bent and/or curved shaft portion 12b includes an angle between 100 degrees and 170 degrees, preferably between 110 degrees and 160 degrees, particularly preferably between 120 degrees and 150 degrees, very particularly preferably between 130 degrees and 140 degrees.

According to the example, the corresponding bend and/or curvature of the bent and/or curved shaft portion 12b includes an angle between 134 degrees and 136 degrees, preferably 135 degrees.

It should also be noted that it may be advantageous if the holding region 13 has a detachable connection, preferably a plug connection and/or a screw connection and/or a bayonet-type connection, in particular a bayonet connection, which is designed to hold the limiting element.

The detachable connection can in particular also be a connection that can be detached by means of a pulling element. Advantageously, the shaft 12 or each of the shaft portions 12a, 12b, 12c can be designed to be hollow, so that the pulling element can be guided in the interior of the shaft 12 or the shaft elements 12a, 12b, 12c, in particular up to the grip region 11. The grip region 11 can preferably have a detaching element which is designed to detach the detachable connection by means of the pulling element or to detach the limiting element from the instrument 10. Preferably, the pulling element may have or be a pulling cable or a pulling wire.

With regard to the holding region 13, it should also be mentioned that it may be advantageous if the holding region 13 is designed to be rotationally symmetrical. According to the example, the holding region 13 is designed to be rotationally symmetrical, which is illustrated in particular by FIG. 4, which shows an enlarged view of the holding region 13.

FIG. 4 additionally shows that the holding region 13 has a thickening 13a. This thickening 13a is designed in particular to hold the limiting element by means of frictional engagement. Additionally or alternatively to the thickening 13a, the holding region 13 can have at least one further thickening and/or at least one O-Ring, in particular for frictionally engaged holding the limiting element.

Before discussing below an exemplary implementation of the system according to the second aspect of the disclosure, for the sake of completeness, reference is made, in particular in the context of the above statements, to an exemplary implementation of the kit in accordance with the third aspect of the disclosure, even if this kit is not explicitly illustrated by means of the figures.

The exemplary implementation of the kit has the instrument 10 described above and at least two shafts 12, each having a different bend and/or curvature, or at least two bent and/or curved shaft portions, each having a different bend and/or curvature.

Accordingly, in particular in the sense of the second of the two aforementioned alternatives, the kit has, for example, the second shaft portion 12b and at least one further second shaft portion, the bend and/or curvature of each of which differs from the bending and/or curvature of the second shaft portion 12b.

FIG. 5 now shows a three-dimensional view of an exemplary implementation 20 of the system according to the disclosure with the instrument 10 described above and a limiting element 30 for limiting the spread of bone cement.

Here, the limiting element 30 is held by means of the holding region 13 of the instrument 10 in particular by appropriate frictional engagement, which is additionally illustrated by the enlarged sectional view of the system 20 with respect to the holding region 13 of the instrument 10 and of the limiting element 30 in FIG. 6.

Further, FIG. 7 and FIG. 8 show a sectional view and a three-dimensional view of the limiting element 30. It can be seen that the limiting element 30 has an interface region 31 which is designed in particular to interact with the holding region 13 of the instrument 10. It could also be said that the interface region 31 is designed such that the limiting element 30 can be held by means of the holding region 13 of the instrument 10, in particular by appropriate frictional engagement.

It should also be mentioned that it may be advantageous if the interface region 31 is designed to be rotationally symmetrical. According to the example, the interface region 31 is designed to be rotationally symmetrical. Further, the limiting element 30 shown by way of example in the exemplary implementation has an axle 33, wherein the limiting element 30 is designed to be rotationally symmetrical in particular with respect to the axle 33.

Preferably, the axle 33 may be a hollow axle having the interface region 31 particularly in the region of a first end of the hollow axle. Further preferably, the hollow axle can be covered and/or closed at a second end, in particular by means of a dome-like or hemispherical cover.

It can be advantageous if the hollow axle in the region of the interface region tapers in its interior at least in portions and/or in steps from the first end toward the second end. According to the example, the taper in the interior of the hollow axle 33 is designed such that it can interact with the thickening 13a in particular in the sense of a frictional engagement.

It may also be advantageous if the limiting element 30 has at least two substantially parallel or parallel disks, preferably at least four substantially parallel or parallel disks, which are arranged in particular equidistantly. According to the example, the limiting element has six substantially parallel or parallel disks 32a, 32b, 32c, 32d, 32e, 32f, which are arranged in particular equidistantly.

Here, the term “substantially parallel” can in particular be understood as meaning that a deviation from the corresponding parallelism or parallels is not more than 15 degrees, preferably not more than 10 degrees, particularly preferably not more than 5 degrees, very particularly preferably not more than 1 degrees.

Preferably, the substantially parallel or fully parallel disks, for example the six substantially parallel or parallel disks 32a, 32b, 32c, 32d, 32e, 32f, are arranged substantially perpendicular or perpendicular to the axle 33.

Here, the term “substantially perpendicular” can in particular be understood as meaning that a deviation from the corresponding orthogonality or perpendicular is not more than 15 degrees, preferably not more than 10 degrees, particularly preferably not more than 5 degrees, very particularly preferably not more than 1 degree.

Preferably, each of the disks, for example each of the six disks 32a, 32b, 32c, 32d, 32e, 32f, has a radius. Further preferably, the axle 33 penetrates at least a portion of the disks, for example each of the six disks, at their respective centers.

It should be noted that it may be advantageous if the respective radius of the disks increases in particular in a continuous manner, with the corresponding increment of the respective increase being preferably constant.

According to the example, the radius of the six disks 32a, 32b, 32c, 32d, 32e, 32f each increases starting from the first disk 32a, which is arranged in the region of the second end of the axle 33, up to the sixth disk 32f, which is arranged in the region of the first end of the axle 33 or in the region of the interface region 31.

Furthermore, it may be advantageous if the limiting element 30, in particular a base material of the limiting element 30, has or consists of polyethylene.

It should be noted that it may also be advantageous if the limiting element 30 is designed fir-tree-like or fir-tree-shaped. Furthermore, at least a part, preferably each of the disks may have spoke-like reinforcements, in particular spokes.

As shown in FIG. 8, in the exemplary implementation, each of the six disks 32a, 32b, 32c, 32d, 32e, 32f of the limiting element 30 each has spoke-like reinforcements 34a, 34b, 34c, 34d, 34e, 34f, in particular spokes.

Although this is not explicitly shown in the figures, it should be noted that it may be advantageous if the limiting element 30, in particular in the region of the interface region 31, has at least one predetermined breaking point, preferably exactly one predetermined breaking point.

In this context, it may be advantageous if the axle 33 or the hollow axle of the limiting element 30 protrudes beyond the disk which is arranged in the region of the interface region 31, for example the sixth disk 32f. Preferably, the protrusion can be formed such that the interface region 31 is free of disks, for example the sixth disk 32f.

With regard to the at least one predetermined breaking point, in particular the exactly one predetermined breaking point, it should be mentioned that it may be particularly advantageous if the at least one or the exactly one predetermined breaking point, preferably in the event that the setting of the limiting element 30 is completed, is designed such that it breaks before the interface region 31 can be detached or is detached from the holding region 13 of the instrument 10, in particular when the instrument 10 is removed.

Finally, with regard to the at least one predetermined breaking point, in particular the exactly one predetermined breaking point, it should be noted that it may be advantageous if the latter is designed to be rotationally symmetrical.

The disclosure is not limited to the exemplary implementations discussed above. All features described in the description or features claimed in the claims or features shown in the drawing can be combined with one another in any way within the scope of this disclosure.

1.-13. (canceled)

14. An instrument for setting a limiting element for limiting the spread of bone cement, the instrument comprising:
a grip region for gripping the instrument;
a hollow shaft having a first end and a second end, the first end of the hollow shaft being coupled to the grip region, the hollow shaft having at least one bend;
a holding region configured to have a detachable connection to the limiting element, the holding region being coupled to the second end of the shaft;
a pulling element disposed within an interior of the hollow shaft, the pulling element extending from the grip region to the holding region; and
a detaching element disposed in the grip region, the detaching element configured to detach the detachable connection by means of the pulling element.

15. The instrument according to claim 14, wherein the hollow shaft has at least two detachably attachable hollow shaft portions.

16. The instrument according to claim 15, wherein the hollow shaft has three detachably attachable hollow shaft portions, including two straight hollow shaft portions and one curved hollow shaft portion.

17. The instrument according to claim 15, wherein the detachable connection comprises a plug connection, a screw connection, a bayonet-type connection, or any combination thereof.

18. The instrument according to claim 14, wherein the hollow shaft has at least two fixedly attached hollow shaft portions, wherein the fixed connection comprises an adhesive connection, a welded connection, a rivet connection, an integral connection, or any combination thereof.

19. The instrument according to claim 14, wherein the first end of the hollow shaft is coupled to the grip region by a detachable connection that comprises a plug connection, a screw connection, a bayonet-type connection, or any combination thereof.

20. The instrument according to claim 14, wherein the first end of the hollow shaft is coupled to the grip region by a fixed connection that comprises an adhesive connection, a welded connection, a rivet connection, an integral connection, or any combination thereof.

21. The instrument according to claim 14, wherein the holding region is coupled to the second end of the shaft by a detachable connection that comprises a plug connection, a screw connection, a bayonet-type connection, or any combination thereof.

22. The instrument according to claim 14, wherein the holding region is coupled to the second end of the shaft by a fixed connection that comprises an adhesive connection, a welded connection, a rivet connection, an integral connection, or any combination thereof.

23. The instrument according to claim 14, wherein the at least one bend includes an angle in a range between bout 100 degrees and 170 degrees.

24. The instrument according to claim 14, wherein the detachable connection of the holding region to the limiting element comprises a plug connection, a screw connection, a bayonet-type connection, or any combination thereof.

25. The instrument according to claim 14, wherein the pulling element by which the detachable connection is detached comprises a cable or a wire.

26. The instrument according to claim 14, wherein the holding region is rotationally symmetrical, and includes at least one thickening for frictionally engaging holding the limiting element.

27. A system comprising:
an instrument for setting a limiting element for limiting the spread of bone cement, the instrument comprising:
a grip region for gripping the instrument;
a hollow shaft having a first end and a second end, the first end of the hollow shaft being coupled to the grip region, the hollow shaft having at least one bend;
a holding region configured to have a detachable connection to the limiting element, the holding region being coupled to the second end of the shaft;
a pulling element disposed within an interior of the hollow shaft, the pulling element extending from the grip region to the holding region; and
a detaching element disposed in the grip region, the detaching element configured to detach the detachable connection by means of the pulling element; and
the limiting element for limiting the spread of bone cement.

28. The system according to claim 27, wherein the limiting element has an interface region that engages with the holding region of the instrument, and wherein the interface region has at least one predetermined breaking point.

29. The system according to claim 27, wherein the limiting element includes an axle and at least two substantially parallel disks that are substantially perpendicular to the axle.

30. The system according to claim 29, wherein the limiting element includes the axle and at least three substantially parallel disks that are arranged equidistantly and substantially perpendicular to the axle.

31. The system according to claim 29, wherein the axle is hollow and includes the interface region in a region of a first end of the hollow axle.

32. The system according to claim 31, wherein the hollow axle is closed at a second end, and wherein the interface region tapers in its interior in steps from the first end of the hollow axle toward the second end.

33. A kit comprising:
the system of clam 27, and
one or more additional hollow shafts, each having a different bend.
